(19) United States
(12) Patent Application Publication
(10) Pub. No.: US 2025/0110106 A1
(43) Pub. Date: Apr. 3, 2025

(54) LITHIUM LOGGING TOOL FOR LITHIUM CONCENTRATION DETERMINATION IN RESERVOIR BRINES

(71) Applicant: SAUDI ARABIAN OIL COMPANY, Dhahran (SA)

(72) Inventors: Klemens Katterbauer, Dhahran (SA); Abdallah A. AlShehri, Dhahran (SA); Abdulaziz Al Qasim, Dammam (SA); Ali Abdullah AlYousif, Dhahran (SA)

(73) Assignee: SAUDI ARABIAN OIL COMPANY, Dhahran (SA)

(21) Appl. No.: 18/476,994

(22) Filed: Sep. 28, 2023

(51) Int. Cl. G01N 33/28; E21B 43/34; E21B 47/10; E21B 47/12; E21B 49/08; G01F 1/05; G01N 1/40

(52) U.S. Cl. CPC G01N 33/2835; E21B 43/34; E21B 47/10; E21B 47/12; E21B 49/08; G01F 1/05; G01N 1/4077; G01N 33/2823; G01N 2001/4083

(57) ABSTRACT

Described is a lithium logging tool. The lithium logging tool includes a sampling and separation module, a lithium concentration module, a microprocessor, and a power module. The sampling and separation module obtains a sample of a fluid from a wellbore and separates hydrocarbons from the sample. The lithium concentration module extracts lithium from the sample. The microprocessor analyzes data related to the fluid in periodic intervals and determines a concentration of lithium in the sample. The power module is configured for providing power to the sampling and separation module and the microprocessor.

### BACKGROUND

Lithium and its compounds are widely used in manufactured glass, ceramics, greases, batteries, refrigerants, chemical reagents and other industries. World lithium reserves are approximately 14 million tons, where 70-80% of lithium reserves are stored in salt lake brine, geothermal water, and solid lithium contained in lithium ore. Lithium demand is expected to grow continuously and dramatically in the coming years as different types of lithium batteries are promising candidates for powering electric and hybrid vehicles. Lithium batteries include battery chemistries, such as lithium-ion, lithium-sulfur, and lithium-air.

Due to the exhaustion of lithium ores, recent studies have described recovery of lithium from seawater, brine, and geothermal water. Production of lithium from water resources has become more important due to water's wide availability, case of process, and cost-effectiveness compared with production from other resources. There are several methods to extract lithium, including solvent extraction, precipitation, liquid-liquid extraction, selective membrane separation, electrodialysis, and ion exchange adsorption. Ion exchange adsorption methods offer significant benefits, such as availability, lower cost, profitability, efficiency, and case of operation. For lithium removal, various lithium adsorbent materials have previously been reported, including metal oxides, clay minerals, silicotitanates, and zirconium phosphate.

Extracting lithium from brine and wastewater from ponds and drilling pits is an important potential resource. When considered from an economic and scientific perspective, the following points are important to consider for lithium recovery from brine: suitability of pond soil and admissibility of the area for solar evaporation; the concentration of lithium in the brine; the ratio of alkali metals and alkaline earth elements to lithium; and the complexity of the phase chemistry. There are three types of resources of brines containing lithium: evaporative, geothermal, and oilfield brines. In the process of evaporation of brine, about 50% of the original natural brine remains. Lithium remains in the residual brine due to the retention of lithium by precipitated salts. Residual brine is highly loaded with magnesium as compared with potassium and sodium, which may make it difficult to extract lithium from the residual brine.

Besides solar evaporation, an alternative source of lithium with concentrations comparable to brines (e.g., 100 to 1,000 mg/L) is the vast volume of produced water generated from oil and gas operations. Although lithium recovery from these wastewater streams is uncommon, the development of new, energy-efficient separation techniques with higher throughput may significantly decrease the cost of isolating lithium from traditional reserves as well as underutilized resources.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a lithium logging tool having a sampling and separation module, a lithium concentration module, a microprocessor, and a power module. The sampling and separation module is configured for obtaining a sample of a fluid from a wellbore and separating hydrocarbons from the sample. The lithium concentration module is configured for extracting lithium from the sample. The microprocessor is configured for analyzing a set of data related to the fluid in periodic intervals and determining a concentration of lithium in the sample. The power module provides power to the sampling and separation module and the microprocessor.

In another aspect, the fluid is reservoir brine.

In another aspect, the lithium logging tool includes a flowmeter configured for measuring flow of the fluid in the wellbore.

In another aspect, the flowmeter is a spinner flowmeter.

In another aspect, the lithium concentration module comprises a ceramic nanofiltration membrane.

In another aspect, the microprocessor is further configured for controlling sampling of the fluid by the sampling and separation module.

In another aspect, the microprocessor is further configured for transmitting the set of data to a surface of the wellbore.

In another aspect, a sampling portion of the sampling and separation module comprises a suction tube configured for obtaining the sample of the fluid.

In another aspect, a separation portion of the sampling and separation module comprises a centrifugal separator.

In one aspect, the embodiments disclosed herein relate to a system including a wellbore, a wireline extending downward into the wellbore, and a lithium logging tool coupled to the wireline.

In one aspect, the embodiments disclosed herein relate to a method for determining a concentration of lithium in a wellbore fluid. A lithium logging tool is lowered into a wellbore. A sample of fluid is obtained from the wellbore, hydrocarbons are separated from the sample, and lithium is extracted from the sample. A set of data related to the fluid is analyzed in periodic intervals, and a concentration of lithium in the sample is determined.

In another aspect, the set of data is transmitted to a surface of the wellbore.

In another aspect, flow of the fluid is measured with a flowmeter of the lithium logging tool.

In another aspect, an amount of lithium in the wellbore is estimated using the concentration of lithium in the sample.

Other aspects and advantages of the claimed subject matter will be apparent from the following description and the appended claims.

### BRIEF DESCRIPTION OF DRAWINGS

FIG. 1 illustrates a well environment according to one or more embodiments of the present disclosure.

FIG. 2 illustrates a downhole lithium logging tool according to one or more embodiments of the present disclosure.

FIG. 3 illustrates a computing system according to one or more embodiments of the present disclosure.

FIG. 4 illustrates a flowchart of a method for lithium concentration determination according to one or more embodiments of the present disclosure.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

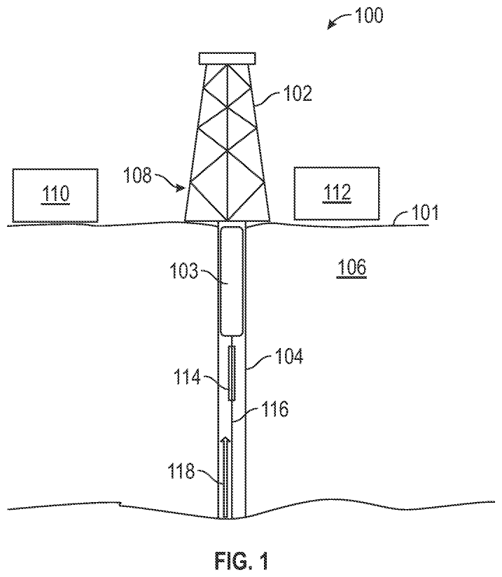

In the following description of FIGS. 1-4, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a passive soil gas sample system” includes reference to one or more of such systems.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

Embodiments disclosed herein include an apparatus and process for determining in situ lithium concentrations in reservoir brine. The apparatus comprises a lithium logging tool configured to obtain and analyze samples of reservoir brine from a formation penetrated by a wellbore. Data related to the sampled reservoir brine is analyzed at the lithium logging tool, since the lithium logging tool integrates a microprocessor and lithium concentration measurement for the determination of lithium concentrations.

FIG. 1 illustrates an exemplary well environment (100) including a well (102) having a wellbore (104) extending into a formation (106). The wellbore (104) may include a bored hole that extends from the surface (101) into a target zone of the formation (106), such as a reservoir. A casing (103) may be installed in the wellbore (104). In one or more embodiments, the casing (103) may be perforated to have perforations into the target formation (106) to allow a flow of reservoir fluid to enter the wellbore (104). The well environment (100) may include a drilling system (108), a logging system (110), and a control system (112). The drilling system (108) may include a drill string, drill bit, a mud circulation system and/or the like for use in boring the wellbore (104) into the formation (106).

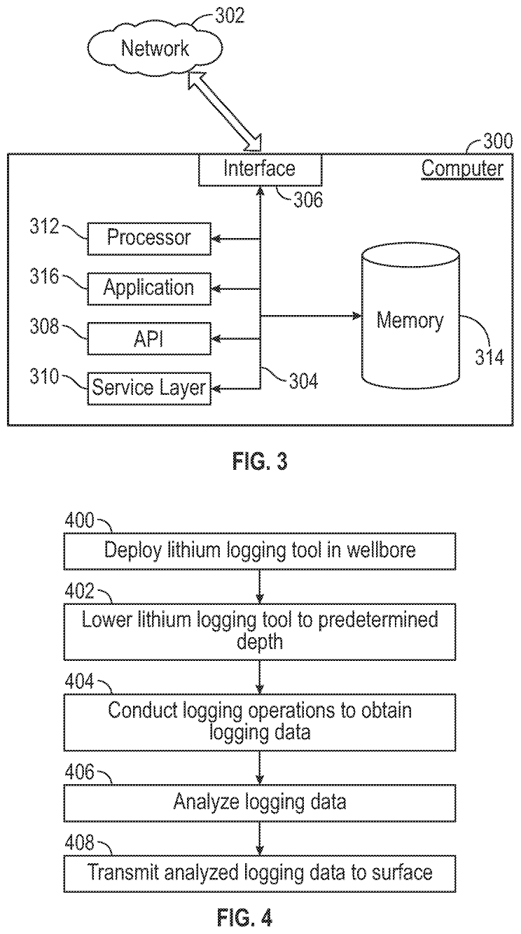

The control system (112) may include hardware and/or software for managing drilling operations and/or maintenance operations. For example, the control system may include one or more programmable logic controllers (PLCs) that include hardware and/or software with functionality to control one or more processes performed by the drilling system (108). Specifically, a programmable logic controller may control valve states, fluid levels, pipe pressures, warning alarms, and/or pressure releases throughout a drilling rig. In particular, a programmable logic controller may be a ruggedized computer system with functionality to withstand vibrations, extreme temperatures, wet conditions, and/or dusty conditions, for example, around a drilling rig. Without loss of generality, the term “control system” may refer to a drilling operation control system that is used to operate and control the equipment, a drilling data acquisition and monitoring system that is used to acquire drilling process and equipment data and to monitor the operation of the drilling process, or a drilling interpretation software system that is used to analyze and understand drilling events and progress. In some embodiments, the control system (112) includes a computing system that is the same as or similar to that of computer (300) described below in FIG. 3 and the accompanying description.

The control system (112) may include functionality to present raw and/or processed data, such as temperature sensor data, flow sensor data, or pressure sensor data, among others. For example, presenting data or alarm states may be accomplished through various presenting methods. Specifically, data or alarm states may be presented through a user interface provided by a computing device. The user interface may include a graphic user interface (GUI) that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a system model. For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data or alarm states may also be presented through various audio methods. In particular, data may be rendered into an audio format and presented as sound through one or more speakers operably connected to a computing device. Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The logging system (110) may include one or more logging tools for use in generating well logs of the formation. For example, a logging tool may be lowered into the wellbore (104) to acquire measurements as the tool traverses a depth interval (e.g., a targeted reservoir section) of the wellbore (104). The plot of the logging measurements versus depth may be referred to as a “log” or “well log”. Well logs may provide depth measurements of the well (102) that describe such reservoir characteristics as formation porosity, formation permeability, resistivity, density, water saturation, and the like. Well logs are correlated with depth based on available well completion reports and well schematics. The resulting logging measurements may be stored and/or processed, for example, by the control system (112), to generate corresponding well logs for the well (102). According to one or more embodiments of the present disclosure, the logging tool is a lithium logging tool (114). The lithium logging tool (114) is lowered into the wellbore (104) to analyze concentrations of lithium in samples of the reservoir brine in the wellbore (104).

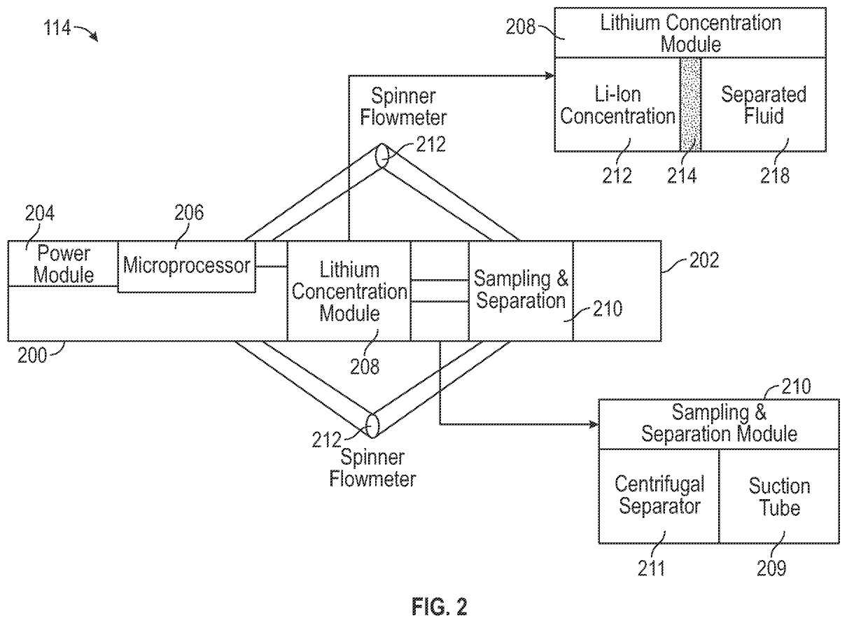

FIG. 2 illustrates the lithium logging tool (114) formed by a string of modules, or tools, interconnected end-to-end together through connections, such as threaded connections and/or electric connections, which couple the modules together. For example, from a first end (200) to a second end (202), the lithium logging tool (114) includes a power module (204) for powering the modules of the tool, a microprocessor (206), a lithium concentration module (208), a sample and separation module (210), and a flowmeter (212). In one or more embodiments, the lithium logging tool (114) is cylindrical, when assembled, and has a length of approximately 10 feet to approximately 15 feet, and a diameter of approximately 1.5 inches to approximately 3⅛ inches.

The lithium logging tool (114) may allow a user to simultaneously collect fluid samples, such as samples of reservoir brine, and log various measurements related to the fluid samples. It is envisioned that the lithium logging tool (114) may collect fluid samples without any time lapse to provide accurate measurements and analysis for data matching to the fluids being logged. The lithium logging tool (114) may include a connector, which is any type of connection element to allow the lithium logging tool (114) to be coupled to a cable (116), such as a wireline, slickline, coiled tubing, or electric line. For example, the connector may be a rope socket connector for the cable to run through a top of the rope socket connector and be locked within the rope socket connector. The rope socket connector may be a wedge type, spool type, split type, camp type or a releasable type of rope socket connector.

The sample and separation module (210) is configured to collect a fluid sample within the wellbore (104), such as reservoir brine. The sample and separation module (210) includes one or more sample chambers. In one or more embodiments, the sample and separation module (210) includes a collection mechanism operationally coupled to the sample and separation module (210). For instance, the collection mechanism may be a piston configured to axially move upward and downward to selectively open and close a sample chamber to collect fluids from the wellbore (104). In one or more embodiments, a length of the piston and the sample and separation module (210) may be extended to hold a plurality of sampling chambers. Further, a movement of the piston may be restricted to a specific depth under a pre-determined voltage to ensure that only one sampling chamber is opened at a time for that pre-determined voltage. Once a second sampling chamber is required to be opened, a second pre-determined voltage may be induced to continue the movement of the piston and open the second sampling chamber. In one or more embodiments, the collection mechanism in a sampling portion of the sampling and separation module (210) is a suction tube (209) that extracts the flowing fluid. The separation portion of the sampling and separation module (210) may be a centrifugal separator (211) that recovers brine from the other hydrocarbons.

The volume of the sample obtained may range from approximately 50 milliliters (ml) to approximately 150 ml. The sampling and separation module (210) is further configured to separate hydrocarbons from the sample of reservoir brine using any suitable approach, such as gravity separation, dispersion, or coalescence. Once hydrocarbons are removed from the sample of reservoir brine in the sampling and separation module (210), the lithium concentration module (208) utilizes a membrane (214) to separate and extract lithium from the hydrocarbon-free fluid, resulting in concentrated lithium (216) and separated fluid (218). Additionally, the lithium concentration module (208) may include at least one probe to transmit lithium concentration data to the surface via the cable (116). In one or more embodiments, the membrane (214) is a ceramic nanofiltration membrane. The ceramic nanofiltration membrane may include a pore size range of 0.1 nanometers (nm) to 10 nm.

The concentration of lithium concentration in the sample may be determined from the lithium concentration module (208), for instance, by laser induced breakdown spectroscopy (LIBS) analysis. Modern technologies, such as handheld devices, may measure lithium as low as approximately 2 parts-per million (ppm) to 5 ppm, such as the Z-300 LIBS system. A lower amount of lithium in a sample of reservoir brine may be approximately 50 ppm to approximately 75 ppm; however, concentrations generally range between approximately 150 ppm and approximately 200 ppm.

The lithium concentration estimates may then be integrated with flow data obtained by the lithium logging tool (114). In one or more embodiments, flow data is obtained from a flowmeter (212) integrated into the lithium logging tool. The flowmeter (212) may be a spinner flowmeter, which measures a flow along the wellbore (104). The spinner may be a fan blade type device that is rotated by fluid movement in the wellbore (104). The speed of rotation of the spinner is then related to the velocity of fluid flow in the wellbore (104). The spinner may be a small diameter type continuous flowmeter, a full-bore type continuous flowmeter, or a diverter type flowmeter. Alternatively, the flowmeter (212) may be an inline flowmeter to measure a flow rate within the wellbore (104). The inline flowmeter may measure a flow rate and a volume of fluid flowing through a bore of the flowmeter (212).

Referring to FIGS. 1 and 2, as fluids are produced from the target formation (106), the reservoir fluids will flow upwards (arrow (118)) through the lithium logging tool (114). For example, the produced fluids may flow through the flowmeter (212), thereby rotating the blade type device to measure a flow rate. Additionally, the produced fluids may flow through the bore of the flowmeter (212) to measure the volume of fluid and the flow rate.

In one or more embodiments, the lithium logging tool (114) includes a microprocessor (206), such as an artificial intelligence (AI) microprocessor, integrated within the lithium logging tool (114). The microprocessor (206) may receive, as input, a set of data including the concentration of lithium in a sample, flow data, and other types of data. For example, the microprocessor (206) may continuously receive measured parameters, such as pressure data, temperature data, optical measurements, electrical fluid measurements, and the like, and integrate the data with the sample point data to estimate an overall lithium concentration for the wellbore (104).

The microprocessor (206) may utilize a deep learning framework, such as a long short-term memory (LSTM) network long short-term memory network (LSTM) framework, to analyze the logging data. A LSTM network is a type of recurrent neural network. The microprocessor (206) may implement a machine learning engine, or model, comprising one or more of an artificial neural network (ANN), a support vector machine, a decision tree, a regression tree (RT), a random forest, an extreme learning machine (ELM), Type I and Type II Fuzzy Logic (T1FL/T2FL), a multivariate linear regression, etc. The machine learning engine may be tuned to match the complexity or otherwise of training data to ensure optimal model performance using adjustable learning parameters, referred to as tuning parameters. The same calibration subset may be used repeatedly while searching for the desired parameters.

The microprocessor (206) may be further configured to control the sampling and separation module (210) such that samples of the reservoir brine are collected in periodic intervals (e.g., every 15 minutes). Additionally, the microprocessor (206) may be configured to automatically analyze and interpret the data as the data is being measured by the various sensors/meters. Alternatively, the collected well fluids may be sent to a lab for analysis.

In one or more embodiments, while running in hole, data from the lithium logging tool (114) is transmitted and analyzed at the surface (101) by the control system (112), in electronic remote communication with the lithium logging tool (114). In one or more embodiments, the data may be transmitted to the surface control system (112) wirelessly. For example, the lithium logging tool (114) may transfer measurement data in real-time uphole via fiber optic communication, which sends pulses of infrared or visible light through an optical fiber. Any combination of mobile, desktop, server, router, switch, embedded device, or other types of hardware may be used. While running in hole and analyzing the well data, real-time decisions may be made in response to the lithium concentrations in the wellbore (104). In one or more embodiments, the decision may be to adjust an injection strategy. For instance, failing to achieve a desired minimum threshold level of lithium (e.g., 50-75 ppm) may indicate that the extraction efficiency is lacking. Therefore, the injection strategy (e.g., continuous gas injection, cyclic gas injection) may be adjusted to increase the lithium extraction efficiency. If real-time data is not needed, data may be stored in a memory and analyzed once the lithium logging tool (114) is pulled to the surface (101).

FIG. 3 further depicts a block diagram of a computer (300) used to provide computational functionalities associated with described algorithms, methods, functions, processes, flows, and procedures as described in this disclosure, according to one or more embodiments. The illustrated computer (300) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (300) may include an input device, such as a keypad, keyboard, touch screen, or other device that can accept user information, and an output device that conveys information associated with the operation of the computer (300), including digital data, visual, or audio information (or a combination of information), or a GUI.

The computer (300) can serve in a role as a client, network component, a server, a database or other persistency, or any other component (or a combination of roles) of a computer system for performing the subject matter described in the instant disclosure. The illustrated computer (300) is communicably coupled with a network (302). In some implementations, one or more components of the computer (300) may be configured to operate within environments, including cloud-computing-based, local, global, or other environment (or a combination of environments).

At a high level, the computer (300) is an electronic computing device operable to receive, transmit, process, store, or manage data and information associated with the described subject matter. According to some implementations, the computer (300) may also include or be communicably coupled with an application server, e-mail server, web server, caching server, streaming data server, business intelligence (BI) server, or other server (or a combination of servers).

The computer (300) can receive requests over network (302) from a client application (for example, executing on another computer (300)) and responding to the received requests by processing the said requests in an appropriate software application. In addition, requests may also be sent to the computer (300) from internal users (for example, from a command console or by other appropriate access method), external or third-parties, other automated applications, as well as any other appropriate entities, individuals, systems, or computers.

Each of the components of the computer (300) can communicate using a system bus (304). In some implementations, any or all of the components of the computer (300), both hardware or software (or a combination of hardware and software), may interface with each other or the interface (306) (or a combination of both) over the system bus (304) using an application programming interface (API) (308) or a service layer (310) (or a combination of the API (308) and service layer (310)). The API (308) may include specifications for routines, data structures, and object classes. The API (308) may be either computer-language independent or dependent and refer to a complete interface, a single function, or even a set of APIs. The service layer (310) provides software services to the computer (300) or other components (whether or not illustrated) that are communicably coupled to the computer (300). The functionality of the computer (300) may be accessible for all service consumers using this service layer. Software services, such as those provided by the service layer (310), provide reusable, defined business functionalities through a defined interface. For example, the interface may be software written in JAVA, C++, or other suitable language providing data in extensible markup language (XML) format or another suitable format. While illustrated as an integrated component of the computer (300), alternative implementations may illustrate the API (308) or the service layer (310) as stand-alone components in relation to other components of the computer (300) or other components (whether or not illustrated) that are communicably coupled to the computer (300). Moreover, any or all parts of the API (308) or the service layer (310) may be implemented as child or sub-modules of another software module, enterprise application, or hardware module without departing from the scope of this disclosure.

The computer (300) includes an interface (306). Although illustrated as a single interface (306) in FIG. 3, two or more interfaces (306) may be used according to particular needs, desires, or particular implementations of the computer (300). The interface (306) is used by the computer (300) for communicating with other systems in a distributed environment that are connected to the network (302). Generally, the interface (306) includes logic encoded in software or hardware (or a combination of software and hardware) and operable to communicate with the network (302). More specifically, the interface (306) may include software supporting one or more communication protocols associated with communications such that the network (302) or interface's hardware is operable to communicate physical signals within and outside of the illustrated computer (300).

The computer (300) includes at least one computer processor (312). Although illustrated as a single computer processor (312) in FIG. 3, two or more processors may be used according to particular needs, desires, or particular implementations of the computer (300). Generally, the computer processor (312) executes instructions and manipulates data to perform the operations of the computer (300) and any algorithms, methods, functions, processes, flows, and procedures as described in the instant disclosure.

The computer (300) also includes a memory (314) that holds data for the computer (300) or other components (or a combination of both) that can be connected to the network (302). For example, memory (314) can be a database storing data consistent with this disclosure. Although illustrated as a single memory (314) in FIG. 3, two or more memories may be used according to particular needs, desires, or particular implementations of the computer (300) and the described functionality. While memory (314) is illustrated as an integral component of the computer (300), in alternative implementations, memory (314) can be external to the computer (300).

The application (316) is an algorithmic software engine providing functionality according to particular needs, desires, or particular implementations of the computer (300), particularly with respect to functionality described in this disclosure. For example, the application (316) can serve as one or more components, modules, applications, etc. Further, although illustrated as a single application (316), the application (316) may be implemented as multiple applications (316) on the computer (300). In addition, although illustrated as integral to the computer (300), in alternative implementations, the application (316) can be external to the computer (300).

There may be any number of computers (300) associated with, or external to, a computer system containing computer (300), wherein each computer (300) communicates over network (302). Further, the term “client,” “user,” and other appropriate terminology may be used interchangeably as appropriate without departing from the scope of this disclosure. Moreover, this disclosure contemplates that many users may use one computer (300), or that one user may use multiple computers (300).

As can be appreciated by one skilled in the art, the lithium logging tool (114) comprises a plurality of sensors and electronics for measuring and transmitting data on the fluids within the wellbore (104) to for the logging data. For instance, the plurality of sensors may include at least one of a pressure sensor, a temperature sensor, a fluid density (radioactive or inertial) sensor, a casing collar locator (CCL) sensor, or any other type of sensor used in production logging operations. The power module (204) may be configured to power, at least, the microprocessor (206) and the sampling and separation module (210) though any suitable power element, such as one or more batteries.

FIG. 4 illustrates a flowchart for using the lithium logging tool (114) in a wellbore (104). One or more steps in FIG. 4 may be performed by one or more components (for example, the computer (300) coupled to the control system (112) in communication with the lithium logging tool (114)) as shown in FIGS. 1 and 2. For example, a non-transitory computer readable medium may store instructions on a memory coupled to a processor such that the instructions include functionality for the lithium logging tool (114).

In step (400), the lithium logging tool is deployed in the wellbore. For example, the lithium logging tool is attached to an end of a cable and is lowered through the wellhead at the surface of the wellhead. Additionally, the cable may be a wireline, slickline, coiled tubing, or electric line to lower and provide power to the lithium logging tool at a depth in the wellbore. In step (402), the lithium logging tool is lowered to a predetermined depth in the wellbore. For example, the predetermined depth may be an area of interest in the wellbore to gather reservoir parameters. Additionally, the predetermined depth may be a depth in the wellbore when the lithium logging tool comes in to contact with fluids produced from a target reservoir.

In step (404), with the lithium logging tool at the predetermined depth, the lithium logging tool conducts production logging operations. For example, fluids in the wellbore rotate the blade type device of spinner of the flowmeter to measure a flow rate. Additionally, the flowmeter of the lithium logging tool may measure the volume of fluid and the flow rate flowing through the bore of the flowmeter. Further, various parameters of fluids are measured by sensors, such as pressure sensors, temperature sensors, fluid density (radioactive or inertial) sensors, casing collar locator (CCL) sensors, and other types of sensors that measure various parameters of the fluids and formation at the predetermined depth. The measurements taken from the lithium logging tool may be stored and transmitted to the surface to form the production logging data.

Embodiments of the present disclosure may provide at least one of the following advantages. The lithium logging tool described herein enables the determination of in-situ lithium concentrations within a well during flow conditions. The lithium logging tool may be sized to various drill-string sizes to accommodate different wells. The determination of lithium concentrations within a well may be used to estimate an amount of lithium that may be recovered from reservoir operations. In one or more embodiments, the lithium concentration may be sampled in periodic intervals, and the average of the lithium is used to estimate the amount of lithium being produced. The concentration per sampled fluid is then multiplied by the overall production of the well.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures.

What is claimed:

1. A lithium logging tool, comprising:
a sampling and separation module configured for:
obtaining a sample of a fluid from a wellbore; and
separating hydrocarbons from the sample;
a lithium concentration module configured for extracting lithium from the sample; and
a microprocessor configured for analyzing a set of data related to the fluid in periodic intervals and determining a concentration of lithium in the sample; and
a power module configured for providing power to the sampling and separation module and the microprocessor.

2. The lithium logging tool of claim 1, wherein the fluid is reservoir brine.

3. The lithium logging tool of claim 1, further comprising a flowmeter configured for measuring flow of the fluid in the wellbore.

4. The lithium logging tool of claim 3, wherein the flowmeter is a spinner flowmeter.

5. The lithium logging tool of claim 1, wherein the lithium concentration module comprises a ceramic nanofiltration membrane.

6. The lithium logging tool of claim 1, wherein the microprocessor is further configured for controlling sampling of the fluid by the sampling and separation module.

7. The lithium logging tool of claim 1, wherein the microprocessor is further configured for transmitting the set of data to a surface of the wellbore.

8. The lithium logging tool of claim 1, wherein a sampling portion of the sampling and separation module comprises a suction tube configured for obtaining the sample of the fluid.

9. The lithium logging tool of claim 1, wherein a separation portion of the sampling and separation module comprises a centrifugal separator.

10. A system, comprising:
a wellbore;
a wireline extending downward into the wellbore; and
a lithium logging tool coupled to the wireline, the lithium logging tool comprising:
a connector coupled to the wireline;
a sampling and separation module configured for:
obtaining a sample of a fluid from the wellbore; and
separating hydrocarbons from the sample;
a lithium concentration module configured for extracting lithium from the sample; and
a microprocessor configured for analyzing a set of data related to the fluid in periodic intervals and determining a concentration of lithium in the sample; and
a power module configured for providing power to the sampling and separation module and the microprocessor.

11. The system of claim 10, wherein the fluid is reservoir brine.

12. The system of claim 10, wherein the lithium logging tool further comprises a flowmeter configured for measuring flow of the fluid.

13. The system of claim 12, wherein the flowmeter is a spinner flowmeter.

14. The system of claim 10, wherein the lithium concentration module comprises a ceramic nanofiltration membrane.

15. The system of claim 10, wherein the microprocessor is further configured to control sampling of the fluid by the sampling and separation module.

16. The system of claim 10, wherein the microprocessor is further configured for transmitting the set of data to a surface of the wellbore.

17. A method for determining a concentration of lithium in a wellbore fluid, comprising:
lowering a lithium logging tool into a wellbore;
obtaining, with a sampling and separation module of the lithium logging tool, a sample of a fluid from the wellbore;
separating, with the sampling and separation module of the lithium logging tool, hydrocarbons from the sample;
extracting, with a lithium concentration module of the lithium logging tool, lithium from the sample;
analyzing, with a microprocessor of the lithium logging tool, a set of data related to the fluid in periodic intervals; and
determining, with the microprocessor of the lithium logging tool, a concentration of lithium in the sample.

18. The method of claim 17, further comprising transmitting the set of data to a surface of the wellbore.

19. The method of claim 17, further comprising measuring flow of the fluid with a flowmeter of the lithium logging tool.

20. The method of claim 17, further comprising using the concentration of lithium in the sample, estimating an amount of lithium in the wellbore.
